(19) United States
(12) Patent Application Publication
(10) Pub. No.: US 2026/0355370 A1
(43) Pub. Date: Nov. 12, 2026

(54) SELF-POWERED LIQUID QUALITY MONITORING AND DISINFECTION STRUCTURE

(71) Applicants: UIF (UNIVERSITY INDUSTRY FOUNDATION), YONSEI UNIVERSITY, Seoul (KR); RESEARCH & BUSINESS FOUNDATION SUNGKYUNKWAN UNIVERSITY, Suwon-si (KR)

(72) Inventors: Sang Woo KIM, Seoul (KR); Dong Min LEE, Seoul (KR); Woo Seok KANG, Paju-si (KR); Min Jae PARK, Seoul (KR); Sung Jin CHO, Suwon-si (KR); Jang Mook JEONG, Suwon-si (KR)

(73) Assignees: UIF (UNIVERSITY INDUSTRY FOUNDATION), YONSEI UNIVERSITY, Seoul (KR); RESEARCH & BUSINESS FOUNDATION SUNGKYUNKWAN UNIVERSITY, Suwon-si (KR)

(21) Appl. No.: 19/652,324

(22) Filed: Apr. 20, 2026

(30) Foreign Application Priority Data
May 9, 2025 (KR) 10-2025-0060609

(51) Int. Cl. A61L 2/03; G01N 33/18; H02K 7/18; H02K 35/02; H02N 1/04; B82Y 5/00

(52) U.S. Cl. CPC A61L 2/03; G01N 33/1826; H02K 7/1884; H02K 35/02; H02N 1/04; A61L 2202/11; A61L 2202/14; B82Y 5/00

(57) ABSTRACT

The present invention relates to a liquid quality management structure capable of self-power generation solely by a user's movement without an external power source. The device includes a coil and a magnet therein to generate power by electromagnetic induction in response to vibration, and has an outer surface structure capable of generating triboelectric charge by friction with a liquid. High aspect ratio nanostructures are formed on the outer surface, and the electric field generated thereby induces electroporation on cell membranes of pathogenic microorganisms to achieve a disinfection effect. The main body is designed with an air layer and a material of similar density to float in the liquid, enabling stable operation in various environments, and includes a sensor for measuring liquid quality and a wireless data transmission module therein, enabling real-time delivery of liquid quality information to a user.

### BACKGROUND

### Field

The present invention relates to a capsule structure capable of monitoring water quality conditions in real time and simultaneously disinfecting using self-powered technology. More particularly, the present invention relates to a portable capsule structure capable of liquid quality monitoring, data transmission, and pathogen disinfection by securing power through triboelectric and electromagnetic induction methods using energy generated from a user's movement without an external power source, and removing pathogenic microorganisms through electroporation technology.

### Description of Related Art

Conventional water quality purification and disinfection technologies have been performed by removing pathogenic microorganisms or contaminants mainly using ultraviolet (UV) irradiation, chemical treatment such as chlorine or hydrogen peroxide, or filtering methods. Although these methods are relatively well-established technologies, there are various limitations in widespread adoption due to inherent drawbacks of each method.

For example, the ultraviolet irradiation method requires continuous power supply for a certain period of time or longer to achieve a disinfection effect, and since precise light source maintenance and high-power output are required, energy consumption is high and maintenance is difficult.

The chemical treatment method has an immediate disinfection effect, but has disadvantages in that there are concerns about secondary contamination caused by residual chemicals, and by-products harmful to the human body or ecosystem may be generated. In addition, the filtering method has limitations in fundamental disinfection due to filter replacement cycles, filtration efficiency, and treatment performance limited only to physical particles.

Meanwhile, in the field of water quality monitoring, most conventional systems rely on stationary wired equipment, and in many cases, separate large-scale equipment or external power sources are required to measure and analyze water quality data. Such systems have low portability, require securing space for device installation, and there is also a risk of contamination of the device itself due to repeated contact with contaminated water. In particular, wired equipment is difficult to install and operate in environments such as remote areas or disaster sites where water resource utilization is limited, making real-time monitoring virtually impossible.

Recently, self-powered technology that generates power through a user's movement without external power supply has been attracting attention, and wearable electronic devices and low-power sensor driving devices using such technology have been partially researched. However, self-powered water quality management technology to date has focused merely on energy harvesting, and has not yet been developed into a convergence-type device that simultaneously performs water quality disinfection and real-time monitoring.

Accordingly, there is a growing need for the development of a portable and self-powered integrated device capable of detecting water quality conditions and effectively removing pathogens without an external power source. The present invention has significance in overcoming the limitations of such conventional technologies and providing a new water quality management technology capable of simultaneously achieving real-time monitoring and high-efficiency disinfection.

### SUMMARY

As used herein, the term "water quality" is not limited to the quality of water, but encompasses the quality condition of any liquid capable of generating triboelectric charge.

An object of the present invention is to overcome various limitations of conventional water quality disinfection and monitoring technologies. Conventional technologies rely on external power, require prolonged operation or high-power equipment, and ultraviolet irradiation or chemical treatment methods involve high energy consumption along with a risk of secondary contamination.

In addition, most water quality monitoring systems have a stationary wired structure, and have problems in that they are bulky and difficult to carry, can only be used in limited environments, and real-time data verification is difficult.

Accordingly, the present invention is intended to implement a structure capable of operating without external power supply by utilizing self-powered energy generated from everyday movements of a user such as walking or shaking, and to provide a portable capsule device capable of monitoring water quality conditions in real time and simultaneously effectively removing pathogenic microorganisms.

In order to achieve the above object, a self-powered liquid quality monitoring and disinfection structure according to an embodiment of the present invention comprises: a main body including a coil and a magnet therein and capable of electromagnetic power generation by movement of the magnet in response to vibration; an outer surface layer surrounding an exterior of the main body and capable of generating triboelectric charge by friction with a liquid; and a surface structure formed on the outer surface layer and configured to electroporate pathogens, wherein the structure is capable of self-power generation by the electromagnetic power generation and the triboelectric charge, and is configured to disinfect the liquid by inactivating and/or removing pathogens therein.

The main body includes a buoyancy structure, and the buoyancy structure includes an air layer between an inner shell and an outer shell of the main body.

The inner shell and the outer shell of the main body are fabricated using a material having a density similar to that of the liquid.

The magnet is capable of electromagnetic power generation in a floating state inside the main body using a magnetic levitation principle.

The outer surface layer includes a metal and an oxide layer formed on a surface of the metal, and the oxide layer serves as a triboelectric inducer and a dielectric. The oxide layer is formed through natural oxidation on the metal.

The surface structure includes a nanostructure having a high aspect ratio. The high aspect ratio is 10:1 or greater.

The main body may further include an electronic circuit unit, and the electronic circuit unit includes a sensor for detecting a quality condition of the liquid; and a wireless communication module.

Within the main body, the electronic circuit unit is spatially separated from a portion performing electromagnetic power generation.

The liquid quality data is transmittable to a user in real time through the wireless communication module.

A self-powered liquid quality monitoring and disinfection capsule according to a further embodiment of the present invention comprises: a main body including an electromagnetic power generation unit including a coil and a magnet therein and capable of electromagnetic power generation by movement of the magnet in response to vibration, and an electronic circuit unit including a sensor for detecting a quality condition of the liquid and a wireless communication module; an outer surface layer surrounding an exterior of the main body and capable of generating triboelectric charge by friction with a liquid; and a surface structure formed on the outer surface layer and configured to electroporate pathogens, wherein the capsule is capable of self-power generation by the electromagnetic power generation and the triboelectric charge, is configured to disinfect the liquid by inactivating and/or removing pathogens therein, and the liquid quality data is transmittable to a user in real time through the wireless communication module.

The main body includes a buoyancy structure, the buoyancy structure includes an air layer between an inner shell and an outer shell of the main body, and the inner shell and the outer shell of the main body are fabricated using a material having a density similar to that of the liquid.

The outer surface layer includes a metal and an oxide layer formed on a surface of the metal, the oxide layer serves as a triboelectric inducer and a dielectric, and the oxide layer is formed through natural oxidation on the metal.

The surface structure includes a nanostructure having a high aspect ratio, and the high aspect ratio is 10:1 or greater.

According to the present invention, by converting mechanical energy generated from everyday movements of a user into power through a self-powered method, liquid quality monitoring and disinfection are possible continuously for an extended period of time without external power supply. Such a self-powered structure can operate stably even in disaster areas, rural areas, remote areas, and the like where power supply is difficult, and thus has high application potential.

In addition, by utilizing a naturally formed oxide layer on a metal surface as a dielectric and destroying cell membranes of pathogenic microorganisms through an electroporation method using an electric field generated by triboelectric charge, high disinfection efficiency can be achieved without chemicals, and environmental safety is greatly improved by minimizing generation of by-products.

Furthermore, since a low-power BLE (Bluetooth Low Energy)-based wireless communication module is embedded, liquid quality conditions can be detected in real time and measurement data can be immediately transmitted to a user, enabling efficient liquid quality management without separate analysis equipment or wired systems. This fundamentally eliminates spatial constraints, electrical exposure, and the possibility of secondary contamination that occurred in conventional systems.

Moreover, the capsule structure of the present invention uses a material having a density similar to that of the liquid and forms an air layer therein to secure buoyancy, so that the device does not sink in the liquid and always operates near the liquid surface, thereby maximizing disinfection efficiency while enhancing convenience of use and portability. As a result, the applicability in various outdoor environments is high, and the present invention exhibits excellent effects as a new liquid quality management solution integrating real-time monitoring and disinfection.

### BRIEF DESCRIPTION OF THE DRAWINGS

FIG. 1 illustrates an exploded assembly view of a self-powered water quality monitoring and disinfection structure according to an embodiment of the present invention.

FIG. 2 illustrates a buoyancy structure of a self-powered water quality monitoring and disinfection structure according to an embodiment of the present invention.

FIG. 3 illustrates a schematic view of a surface structure using nanorods.

FIG. 4 illustrates a schematic diagram of a real-time liquid quality monitoring and disinfection system using self-power generation.

FIG. 5 illustrates an operation example of a self-power generation-based liquid quality monitoring and disinfection system utilizing everyday movements.

FIG. 6 illustrates a comparison of triboelectric output between a submerged state and a floating state of the structure according to the present invention in water.

FIG. 7 illustrates an evaluation of output voltage and current characteristics according to the number of coil windings and vibration frequency of the internal electromagnetic power generation system.

FIG. 8 illustrates an overall system configuration diagram of a self-powered liquid quality monitoring and disinfection device using triboelectric and electromagnetic induction according to the present invention.

FIG. 9 illustrates a comparative conceptual diagram of a conventional water quality monitoring and disinfection process and a self-powered liquid quality monitoring and disinfection system according to the present invention.

Various embodiments will now be described with reference to the drawings, and like reference numerals are used throughout the drawings to designate like elements. For purposes of explanation, various descriptions are presented herein to provide an understanding of the present invention. However, it is apparent that these embodiments may be practiced without such specific descriptions. In other examples, well-known structures and devices are presented in block diagram form to facilitate description of the embodiments.

### DETAILED DESCRIPTIONS

Hereinafter, embodiments of the present invention will be described in detail with reference to the accompanying drawings. Since the present invention may be subject to various modifications and may have various forms, specific embodiments are illustrated in the drawings and described in detail herein. However, this is not intended to limit the present invention to specific disclosed forms, and should be understood to include all modifications, equivalents, and alternatives included within the spirit and technical scope of the present invention. Like reference numerals have been used for like components in describing each drawing.

The terminology used in this application is used only to describe specific embodiments and is not intended to limit the present invention. Singular expressions include plural expressions unless the context clearly dictates otherwise. In this application, the terms "comprise" or "have" are intended to designate that a feature, step, operation, component, part, or a combination thereof described in the specification exists, and should be understood not to preclude the existence or addition possibility of one or more other features, steps, operations, components, parts, or combinations thereof.

The present invention includes a capsule structure in which buoyancy is secured by forming an air layer between an inner shell and an outer shell fabricated from a material having a density similar to that of a liquid (e.g., water), and a self-powered system using a magnet and a coil is mounted inside the capsule structure to generate power by electromagnetic power generation through movement or vibration. In addition, triboelectric charge may be additionally generated on a metal surface of the outer shell on which an oxide layer is formed. The self-generated power is utilized for disinfecting pathogenic microorganisms (pathogens) through an electroporation method, and the present invention discloses a self-powered liquid quality management device that simultaneously performs a monitoring function by measuring liquid quality conditions in real time through an electronic circuit unit including a sensor and wirelessly transmitting data.

As used herein, the term "liquid" refers to any liquid medium capable of generating triboelectric charge upon contact with the outer surface layer, and includes, but is not limited to, water, aqueous solutions, saline solutions, other polar solvents, viscous liquids, chemical solutions, and mixtures thereof. In addition, as used herein, the term "water quality" refers to a quality condition of the liquid, and shall be construed to encompass not only the quality of water but also condition parameters of the liquid as defined above, such as conductivity, temperature, and pH.

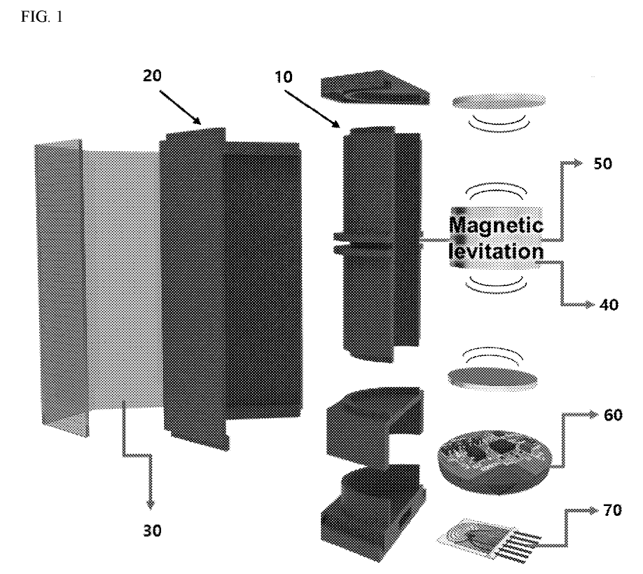

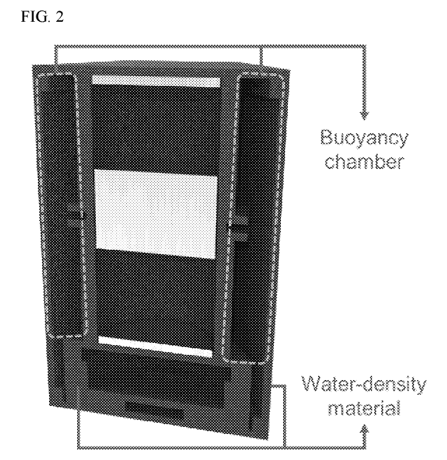

FIG. 1 illustrates an exploded assembly view of a self-powered water quality monitoring and disinfection structure according to an embodiment of the present invention. FIG. 2 illustrates a buoyancy structure of a self-powered water quality monitoring and disinfection structure according to an embodiment of the present invention.

A self-powered water quality monitoring and disinfection structure according to an embodiment of the present invention includes a main body (10); an outer surface layer (20) surrounding an exterior of the main body; and a surface structure (30) formed on the outer surface layer.

The main body (10) includes a coil and a magnet therein and has a form capable of electromagnetic power generation by movement of the magnet in response to vibration or movement.

As shown in FIG. 2, the main body includes a buoyancy structure, and the buoyancy structure includes an air layer between an inner shell and an outer shell of the main body.

In this case, the inner shell and the outer shell of the main body are fabricated using a material having a density similar to that of the liquid, and buoyancy is secured by securing an air layer therein.

In this case, the material having a density similar to that of the liquid includes, for example, nylon or polypropylene.

As shown in FIG. 2, by including a separate air layer between the inner shell and the outer shell, the structure is designed so that the heavy magnet can float on the liquid by buoyancy. By designing the structure such that the structure (capsule) of the present invention does not sink in the liquid and can efficiently contact the liquid near the liquid surface, a higher disinfection effect of the liquid can be achieved compared to when submerged in the liquid, the structure can be usefully used in various locations, and sensing and disinfection performance for liquid quality can be stably maintained.

In summary, by designing the capsule structure to stably float near the liquid surface utilizing a buoyancy-providing structure and a material having a density similar to that of the liquid (e.g., water), contact and separation between the liquid and the electrode are more smoothly achieved, and through this, the triboelectric effect described below is expected to be maximized, thereby also improving disinfection performance.

As shown in FIG. 2, a coil (50) and a magnet (40) are disposed inside the main body, and the coil (50) is wound on an inner surface of the main body, and for example, an enamel-coated copper wire or the like may be used.

The magnet (40) is disposed to be movable along one axis inside the main body as shown in FIG. 2. For example, in FIG. 2, a space in which the magnet (40) can move vertically up and down is formed along the space inside the main body, so that the magnet is movable up and down in response to external movement or vibration. For example, a neodymium magnet may be used as the magnet.

Power can be generated by electromagnetic power generation inside the main body by such movement of the magnet.

Meanwhile, the magnet (40) is capable of electromagnetic power generation in a floating state inside the main body using a magnetic levitation principle. That is, by maintaining the magnet in a floating state in the air inside the capsule using the magnetic levitation principle, mechanical friction is minimized so that the durability of the device is excellent, and power generation efficiency can be increased by sensitively responding to even small external vibrations. This provides an advantage of dramatically improving energy harvesting efficiency by effectively overcoming the disadvantage of conventional electromagnetic power generation devices in which energy loss is significant due to internal friction.

The outer surface layer (20) surrounds an exterior of the main body and is capable of generating triboelectric charge by friction with the liquid. An air layer may be included between the outer surface layer (outer shell) and an outer surface of the main body (inner shell).

The outer surface layer (20) includes a metal and an oxide layer formed on a surface of the metal.

The oxide layer serves as a triboelectric inducer and a dielectric, and specifically, by utilizing the oxide layer on the metal surface as a dielectric layer, electrical energy can be effectively delivered without occurrence of a short circuit even upon direct contact with the liquid.

In this case, triboelectric charge is generated by friction between the liquid and the surface oxide layer (surface coating layer) in the liquid (e.g., water), and static electricity is generated by a charging phenomenon between the liquid and the metal material inside the oxide layer, thereby generating an alternating electric field in the surroundings. Specifically, when friction occurs between the oxide layer and the liquid, triboelectric static electricity is induced through contact and separation, and a charging phenomenon occurs in the metal and the liquid, thereby generating an alternating electric field around the device.

The method of utilizing the oxide layer formed on the outer metal surface as a dielectric secures stability by preventing a short circuit that may occur upon direct contact with the liquid without separate application of insulating material or additional manufacturing processes. As a result, effects of time and cost reduction in the manufacturing process can be simultaneously realized.

The oxide layer is formed through natural oxidation on the metal, and representative metals and oxides are as follows.

Aluminum (Al) and aluminum oxide (Al2O3) have high dielectric constant and insulating properties, enabling stable electric field delivery without electric field loss. Titanium (Ti) and titanium oxide (TiO2) have excellent biocompatibility and maintain stable dielectric characteristics even in an aqueous environment. Zinc (Zn) and zinc oxide (ZnO) are dielectrics having semiconductor characteristics and have high electrochemical stability in an aqueous environment.

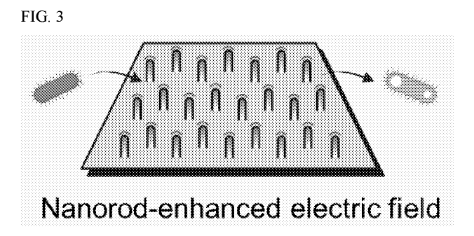

The surface structure (30) is formed on the outer surface layer (20) and is a portion configured to electroporate pathogens. The present invention is based on a principle of disinfection using electroporation in which an electric field of approximately 1V level is applied to pathogenic microorganisms to form microscopic pores in cell membranes. That is, the device surface of the present invention includes a nanostructure that facilitates contact and separation with the liquid, and achieves a disinfection effect by destroying cell membranes of pathogenic microorganisms through an electroporation method using the electric field generated thereby. FIG. 3 illustrates a schematic view of a surface structure using nanorods.

In this case, the surface structure includes high aspect ratio nanostructures such as nanowires or nanorods. When such high aspect ratio structures are formed on the surface, a larger contact area is provided even with the same movement, enabling formation of a stronger localized electric field. In addition, such high aspect ratio nanostructures have an effect of concentrating the electric field at a nanoscale, enabling effective electroporation even with small energy.

An aspect ratio of approximately 10:1 or greater can sufficiently exhibit an electric field concentration effect, and preferably, at a level of 30:1 to 100:1, electric field concentration occurs more strongly, so that a disinfection effect with high energy efficiency can be expected.

Meanwhile, regarding the shape of the nanostructure having an aspect ratio, even if the aspect ratio is somewhat low, when the tip of the structure has a pointed shape (e.g., pyramid-type, needle-type), a field enhancement effect occurs at the tip, forming a strong localized electric field. As such, pointed-shape nanostructures effectively amplify the electric field induced from triboelectric charge, thereby helping to easily exceed the threshold value required for electroporation.

As described above, the self-powered water quality monitoring and disinfection structure according to an embodiment of the present invention is capable of self-power generation by electromagnetic power generation and the triboelectric charge, and is configured to disinfect the liquid by inactivating and/or removing pathogens therein.

Meanwhile, as illustrated in FIG. 1, the self-powered water quality monitoring and disinfection structure according to an embodiment of the present invention may further include an electronic circuit unit (60) in the main body. The electronic circuit unit may include a sensor (70) for detecting a quality condition of the liquid; and a wireless communication module (not shown). Liquid quality data is transmittable to a user in real time through the wireless communication module.

As illustrated in FIGS. 1 and 2, the electronic circuit unit may be spatially separated from a portion performing electromagnetic power generation inside the main body. The internal configuration of the device may broadly include a low-power electronic circuit and an electromagnetic generator, and the low-power electronic circuit is composed of sensors such as a conductivity sensor serving as an indicator of liquid quality condition, and a BLE wireless communication module, so that liquid quality data measured in real time can be delivered to a user.

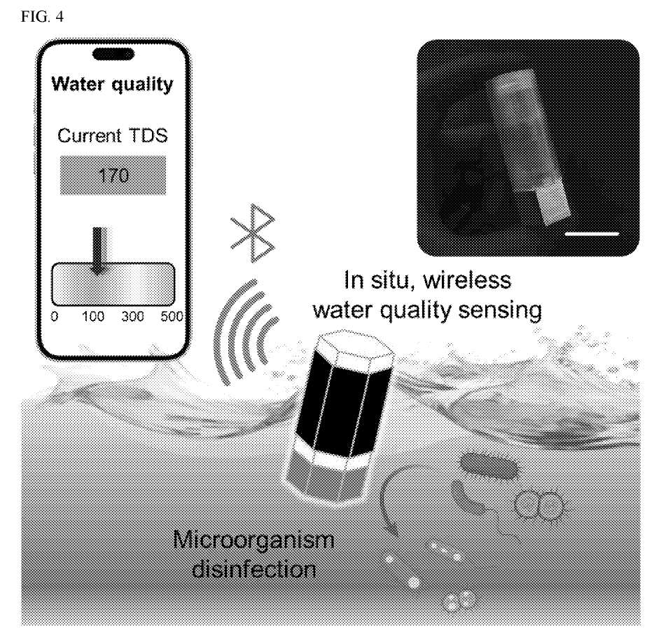

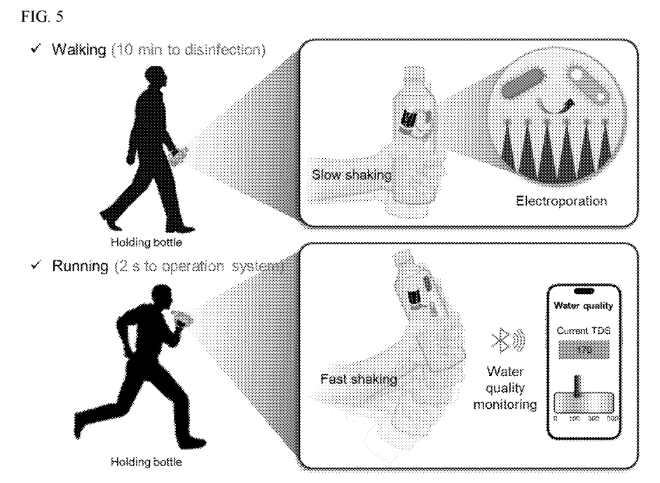

FIG. 4 illustrates a schematic diagram of a real-time liquid quality monitoring and disinfection system using self-power generation, and FIG. 5 illustrates an operation example of a self-power generation-based liquid quality monitoring and disinfection system utilizing everyday movements.

As shown in FIG. 5, when the structure or capsule according to an embodiment of the present invention is placed in a user's water bottle and the user shakes it or carries it in a bag while moving, power is generated by electromagnetic induction due to the movement of the magnet and by triboelectric charge due to friction with the liquid, and the generated power can be utilized for driving the sensor and the BLE module.

As a result, the present invention provides a complete self-powered portable liquid quality management solution that simultaneously realizes liquid quality monitoring and disinfection, by generating triboelectric charge through contact of the device's outer surface with the liquid and disinfecting microorganisms using an electroporation method, while also rapidly sensing the quality condition of the liquid with power generated through electromagnetic power generation by the movement of the magnet and coil inside the device and immediately notifying the user through BLE wireless communication.

A self-powered water quality monitoring and disinfection capsule according to a further embodiment of the present invention comprises: a main body including an electromagnetic power generation unit including a coil and a magnet therein and capable of electromagnetic power generation by movement of the magnet in response to vibration, and an electronic circuit unit including a sensor for detecting a quality condition of the liquid and a wireless communication module; an outer surface layer surrounding an exterior of the main body and capable of generating triboelectric charge by friction with a liquid; and a surface structure formed on the outer surface layer and configured to electroporate pathogens, wherein the capsule is capable of self-power generation by the electromagnetic power generation and the triboelectric charge, is configured to disinfect the liquid by inactivating and/or removing pathogens therein, and liquid quality data is transmittable to a user in real time through the wireless communication module.

In this case, the main body includes a buoyancy structure, the buoyancy structure includes an air layer between an inner shell and an outer shell of the main body, and the inner shell and the outer shell of the main body are fabricated using a material having a density similar to that of the liquid.

The outer surface layer includes a metal and an oxide layer formed on a surface of the metal, the oxide layer serves as a triboelectric inducer and a dielectric, and the oxide layer is formed through natural oxidation on the metal.

The surface structure is formed on the outer surface layer, and the surface structure includes a nanostructure having a high aspect ratio.

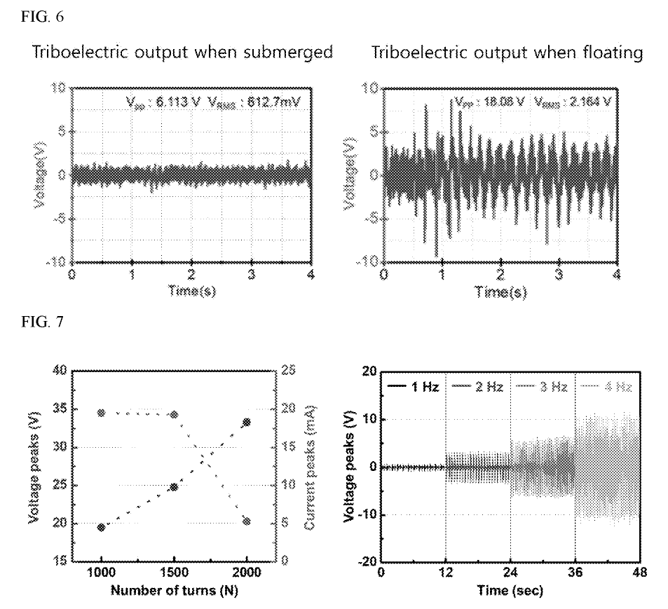

FIG. 6 illustrates a comparison of triboelectric output between a submerged state and a floating state of the structure according to the present invention in water.

As a result of measuring the triboelectric output generated when a device having high aspect ratio nanostructures formed on the surface was placed in water and shaken, it was confirmed that an RMS voltage of 1V or greater was stably formed, which indicates a sufficient electric field for effectively disinfecting pathogenic microorganisms through electroporation, and excellent disinfection effects were also verified in additionally conducted disinfection performance evaluations.

FIG. 7 illustrates an evaluation of output voltage and current characteristics according to the number of coil windings and vibration frequency of the internal electromagnetic power generation system. FIG. 7 evaluates the performance of the electromagnetic power generation system inside the self-powered capsule, and shows a tendency in which voltage increases and current decreases according to the number of coil windings. In addition, as the vibration frequency increases, the amplitude of the output voltage increases, indicating that higher power generation is possible with larger movements. Through this, design criteria for adjusting self-power generation efficiency are presented.

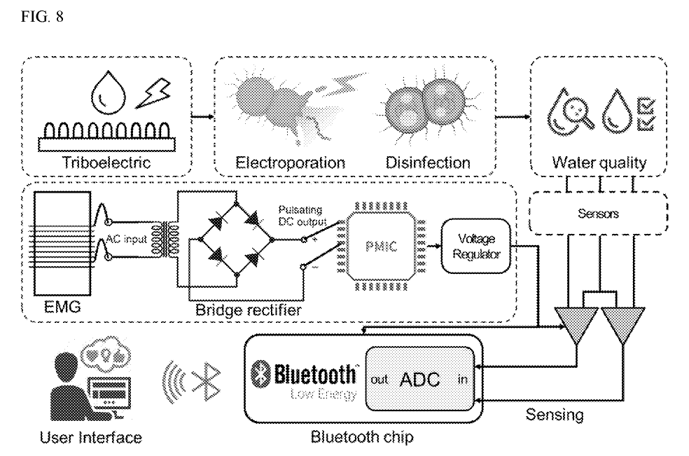

FIG. 8 illustrates an overall system configuration diagram of a self-powered liquid quality monitoring and disinfection device using triboelectric and electromagnetic induction according to the present invention. The electric field generated through triboelectric charge induces electroporation on cell membranes of pathogenic microorganisms for disinfection, and the alternating current generated through electromagnetic induction is supplied to the BLE sensor circuit through a rectification and power management integrated circuit (PMIC). The sensor detects liquid quality conditions (e.g., conductivity, temperature, etc.), and transmits data to the user in real time through BLE wireless communication. This configuration realizes a self-powered smart liquid quality management system capable of simultaneously performing liquid quality disinfection and monitoring without external power.

The sensor mounted on the device can measure various parameters indicating liquid quality conditions, such as electrical conductivity, in real time, and the measured data is immediately transmitted to an external electronic device through BLE wireless communication to provide real-time monitoring information to the user. In addition, by replacing or additionally attaching sensors to detect various liquid quality parameters such as temperature and pH in addition to electrical conductivity, the measurement items of the device can be easily expanded, enabling flexible response to various environments.

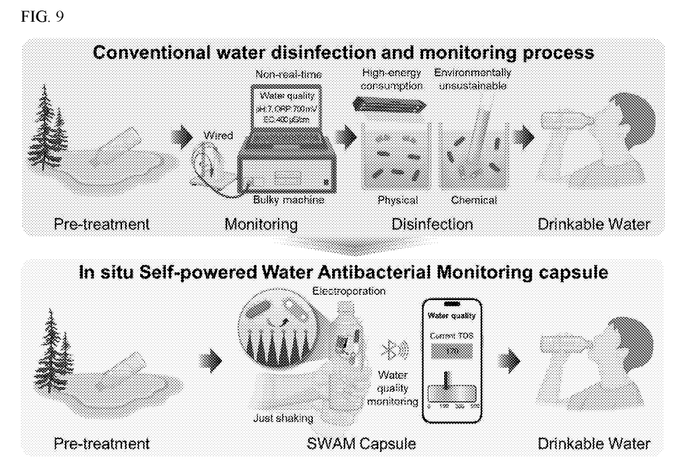

FIG. 9 illustrates a comparative conceptual diagram of a conventional water quality monitoring and disinfection process and a self-powered liquid quality monitoring and disinfection system according to the present invention. The conventional system relies on wired equipment and chemical/physical treatment methods, showing limitations in high energy consumption and environmental sustainability. In contrast, the SWAM capsule of the present invention enables electroporation-based disinfection and real-time wireless monitoring with simple shaking alone, presenting an innovative alternative that satisfies portability, eco-friendliness, and immediacy.

Although the present invention has been described above with reference to preferred embodiments thereof, it will be understood by those skilled in the art that various modifications and changes can be made to the present invention without departing from the spirit and scope of the present invention as set forth in the following claims.

What is claimed is:

1. A self-powered liquid quality monitoring and disinfection structure comprising:
a main body including a coil and a magnet therein and capable of electromagnetic power generation by movement of the magnet in response to vibration;
an outer surface layer surrounding an exterior of the main body and capable of generating triboelectric charge by friction with a liquid; and
a surface structure formed on the outer surface layer and configured to electroporate pathogens,
wherein the structure is capable of self-power generation by the electromagnetic power generation and the triboelectric charge, and is configured to disinfect the liquid by inactivating and/or removing pathogens therein.

2. The self-powered liquid quality monitoring and disinfection structure of claim 1, wherein the main body includes a buoyancy structure, and the buoyancy structure includes an air layer between an inner shell and an outer shell of the main body.

3. The self-powered liquid quality monitoring and disinfection structure of claim 2, wherein the inner shell and the outer shell of the main body are fabricated using a material having a density similar to that of the liquid.

4. The self-powered liquid quality monitoring and disinfection structure of claim 1, wherein the magnet is capable of electromagnetic power generation in a floating state inside the main body using a magnetic levitation principle.

5. The self-powered liquid quality monitoring and disinfection structure of claim 1, wherein the outer surface layer includes a metal and an oxide layer formed on a surface of the metal, and the oxide layer serves as a triboelectric inducer and a dielectric.

6. The self-powered liquid quality monitoring and disinfection structure of claim 5, wherein the oxide layer is formed through natural oxidation on the metal.

7. The self-powered liquid quality monitoring and disinfection structure of claim 1, wherein the surface structure includes a nanostructure having a high aspect ratio.

8. The self-powered liquid quality monitoring and disinfection structure of claim 7, wherein the high aspect ratio is 10:1 or greater.

9. The self-powered liquid quality monitoring and disinfection structure of claim 1, wherein the main body further includes an electronic circuit unit.

10. The self-powered liquid quality monitoring and disinfection structure of claim 9, wherein the electronic circuit unit includes a sensor for detecting a quality condition of the liquid; and a wireless communication module.

11. The self-powered liquid quality monitoring and disinfection structure of claim 9, wherein the electronic circuit unit is spatially separated from a portion performing electromagnetic power generation within the main body.

12. The self-powered liquid quality monitoring and disinfection structure of claim 10, wherein liquid quality data is transmittable to a user in real time through the wireless communication module.

13. A self-powered liquid quality monitoring and disinfection capsule comprising:
a main body including an electromagnetic power generation unit including a coil and a magnet therein and capable of electromagnetic power generation by movement of the magnet in response to vibration, and an electronic circuit unit including a sensor for detecting a quality condition of the liquid and a wireless communication module;
an outer surface layer surrounding an exterior of the main body and capable of generating triboelectric charge by friction with a liquid; and
a surface structure formed on the outer surface layer and configured to electroporate pathogens,
wherein the capsule is capable of self-power generation by the electromagnetic power generation and the triboelectric charge, is configured to disinfect the liquid by inactivating and/or removing pathogens therein, and liquid quality data is transmittable to a user in real time through the wireless communication module.

14. The self-powered liquid quality monitoring and disinfection capsule of claim 13, wherein the main body includes a buoyancy structure, the buoyancy structure includes an air layer between an inner shell and an outer shell of the main body, and the inner shell and the outer shell of the main body are fabricated using a material having a density similar to that of the liquid.

15. The self-powered liquid quality monitoring and disinfection capsule of claim 13, wherein the outer surface layer includes a metal and an oxide layer formed on a surface of the metal, the oxide layer serves as a triboelectric inducer and a dielectric, and the oxide layer is formed through natural oxidation on the metal.

16. The self-powered liquid quality monitoring and disinfection capsule of claim 13, wherein the surface structure includes a nanostructure having a high aspect ratio, and the high aspect ratio is 10:1 or greater.
